(19) United States
(12) Patent Application Publication
(10) Pub. No.: US 2004/0171929 A1
(43) Pub. Date: Sep. 2, 2004

(54) Method and device for determining the contour of a recess in a piece of material

(75) Inventors: Francois Leitner, Uriage (FR); Benoit Mollard, Echirolles (FR); Hanns-Peter Tuemmler, Tuttlingen (DE)

Correspondence Address: LAW OFFICE OF BARRY R LIPSITZ, 755 MAIN STREET, MONROE, CT, 06468, US

(73) Assignee: AESCULAP AG & Co. KG, Tuttlingen (DE)

(21) Appl. No.: 10/632,693

(22) Filed: Jul. 31, 2003

Related U.S. Application Data
(63) Continuation of application No. PCT/EP02/00793, filed on Jan. 25, 2002.

(30) Foreign Application Priority Data
Feb. 7, 2001 (DE) 101 05 822.5

(51) Int. Cl. A61B 19/00; A61B 5/05

(52) U.S. Cl. 600/424; 606/130

(57) ABSTRACT

To enable the contour of a recess in a piece of material, which is worked into the material by means of a tool, in particular in a piece of bone, to also be determined without a direct view of the machining region with a method for determining this contour, it is proposed that the position of the piece of material in space is established by a navigation system, that the position of the tool in space is established by a navigation system, that the respective position of the tool in relation to the piece of material is determined from the position data obtained in this manner, that during the machining of the piece of material with the tool, the relative positions of the tool in relation to the piece of material are stored and that the prepared contour on the piece of material is determined from extreme values of these relative positions with respect to a fixed reference position of the piece of material. A device for performing this method is also specified.

The present disclosure relates to the subject matter disclosed in international application PCT/EP 02/00793 of Jan. 25, 2002, which is incorporated herein by reference in its entity and for all purposes.

### BACKGROUND OF THE INVENTION

The invention relates to a method for determining the contour of a recess in a piece of material, which is worked into the material by means of a tool, in particular in a piece of bone.

It is frequently necessary to incorporate a recess of a determined shape and size into a piece of material, e.g. in a piece of bone, in which the recess is prepared to receive an implant. It is important in this case to obtain the given contour of the recess and not to overstep this given contour.

Such recesses are generally made with special tools, e.g. with rotary profiling cutters, with end mills, drills or similar tools, but also with hand-guided tools, e.g. scalpels, scarifiers, files etc. In practice, it is extremely difficult to look in the worked recesses themselves and see there the course of the contour that has been respectively produced by the tool. This is rendered difficult by the removed material and additionally because the recess is frequently difficult to access or is not accessible at all, and therefore cannot be observed. In these cases, it is left to touch to match the size and shape of the recess as closely as possible to the desired contour.

### SUMMARY OF THE INVENTION

An object of the invention is to provide a method, which enables the actual contour of the recess made with the tool to be determined even when the recess cannot be directly observed.

This object is achieved according to the invention with a method of the above-described type in that the position of the piece of material in space is established by a navigation system, that the position of the tool in space is established by a navigation system, that the respective position of the tool in relation to the piece of material is determined from the position data obtained in this manner, that during the machining of the piece of material with the tool, the relative positions of the tool in relation to the piece of material are stored and that the prepared contour on the piece of material is determined from extreme values of these relative positions with respect to a fixed reference position of the piece of material.

Therefore, in this method both the position of the piece of material and the position of the tool in space are firstly continuously determined during the entire machining process. This may be achieved by using a so-called navigation system. These are systems known per se, which enable the position of an object in space to be precisely determined. For example, both on the piece of material and on the tool, marking elements, e.g. three or even more marking elements, can be fastened which serve as transmitters or reflectors for an electromagnetic radiation and which reflect the electromagnetic radiation emitted from a stationary navigation system and transmit this back to the navigation system, or emit such a radiation and relay this to the navigation system. From the differences in transit time of the radiation relayed via a plurality of transmitters and receivers, the precise positions of the different marking elements on the piece of material or on the tool can be determined, and the exact position of the piece of material and the tool can be determined therefrom.

These position data also enable the relative position of the tool and the piece of material to be determined. In this case, the tool has a specific geometry of the machining areas, e.g. of a semispherical profiling cutter, and this geometry is always the same relative to the marking element fastened to the tool. Therefore, it is possible to determine the position of the machining surface of the tool in relation to the piece of material to be machined from the determination of the position of the marking element, namely continuously during the entire machining process.

These position data, which specify the relative position of the tool and piece of material, are stored over the entire machining process. From these data, extreme positions of the tool in relation to the piece of material can be determined, namely in relation to a fixed position of the piece of material. In other words, working from a fixed point of the piece of material, for example, it is possible to determine in a specific direction when the machining surface of the tool is at a maximum distance from this point. This distance then corresponds to the actual contour of a recess worked into the piece of material, since the tool always machines the material of the piece of material as far as this extreme position. When the distance from the fixed point is less, the machining surface of the tool does not touch the material of the piece of material, nor does it remove any material.

Such a determination can be achieved in different directions working from the fixed reference position, and in this way over the entire surface of the machined recess information is obtained as to where the boundary face between the recess and the material actually runs, i.e. to what extent the tool has respectively removed material from the piece of material.

These data enable the operator to obtain precise information concerning the respective position and size of the recess, even if he/she cannot observe the recess itself.

It is advantageous in this case if the difference of the contour of the worked recess determined in this manner from a given contour is determined at different locations of the contour.

This method works from a desired contour of the recess, whose position data relative to the piece of material can also be stored. These position data of the desired contour are continuously compared with the position data of the actually prepared contour of the recess and reveal whether further material should be removed in order to obtain the desired contour, whether the desired contour has already been obtained or whether the desired contour has in fact been overstepped.

In a particularly preferred embodiment it is provided that the differences determined in this manner at different locations of the prepared recess are visually displayed, wherein it is advantageous if the same differences at different locations of the prepared recess are displayed in the same manner, in particular in the same colour. Therefore, from such a display the operator can directly determine which regions of the recess still require machining in order to obtain the desired contour, in which regions this has already been achieved, and in which regions the desired contour has possibly been overstepped.

It is also advantageous if an image of the piece of material is superposed on the image of the contour of the recess or of the detected difference. This image can provide the operator with information about the surrounding area of the recess so that the operator can establish precisely in which region of the piece of material he/she is working and whether he/she is possibly working into structures of the piece of material, which should not be reached from the recess.

In particular, it can be provided that from the determined extreme values of the relative position, a warning signal is generated when these extreme values exceed specific given maximum values, i.e. when the actually prepared recess threatens to penetrate into regions of the piece of material, into which penetration is not desirable. This can be of importance, for example, if a recess is worked into a bone to receive an implant and if it should be assured that this recess does not exceed a determined size, for instance, to prevent the bone from being broken through at the location of the recess.

An object of the invention is also to propose a device for determining the contour of a recess in a piece of material with a tool for working the contour into the piece of material, with which the operator is able to monitor the contour of the machined recess.

This object is achieved according to the invention with a device of the above-described type in that the piece of material and the tool are respectively firmly connected to a marking element, that a stationary navigation system is provided, which establishes the position of the two marking elements and thus the position of the piece of material and the tool in space, that an arithmetic unit with memory is provided, which from the position data obtained in this manner determines the respective position of the tool in relation to the piece of material, stores the relative positions of the tool in relation to the piece of material still during machining of the piece of material by the tool, and determines the prepared contour on the piece of material from extreme values of these relative positions with respect to a fixed reference position of the piece of material.

Further advantageous configurations of the devices are the subject of sub-claims.

### BRIEF DESCRIPTION OF THE DRAWINGS

The following description of preferred embodiments of the invention serves for more detailed explanation in association with the drawing:

FIG. 1 shows the machining of a piece of material in the form of a hip bone with a cutting tool using a navigation system;

FIG. 2 is a sectional view through a cup-shaped recess in a hip bone;

FIG. 3 is a view similar to FIG. 2 with cutting tools represented in broken lines; and

FIG. 4 is a screen display of a cup-shaped recess showing wall regions removed to different depths.

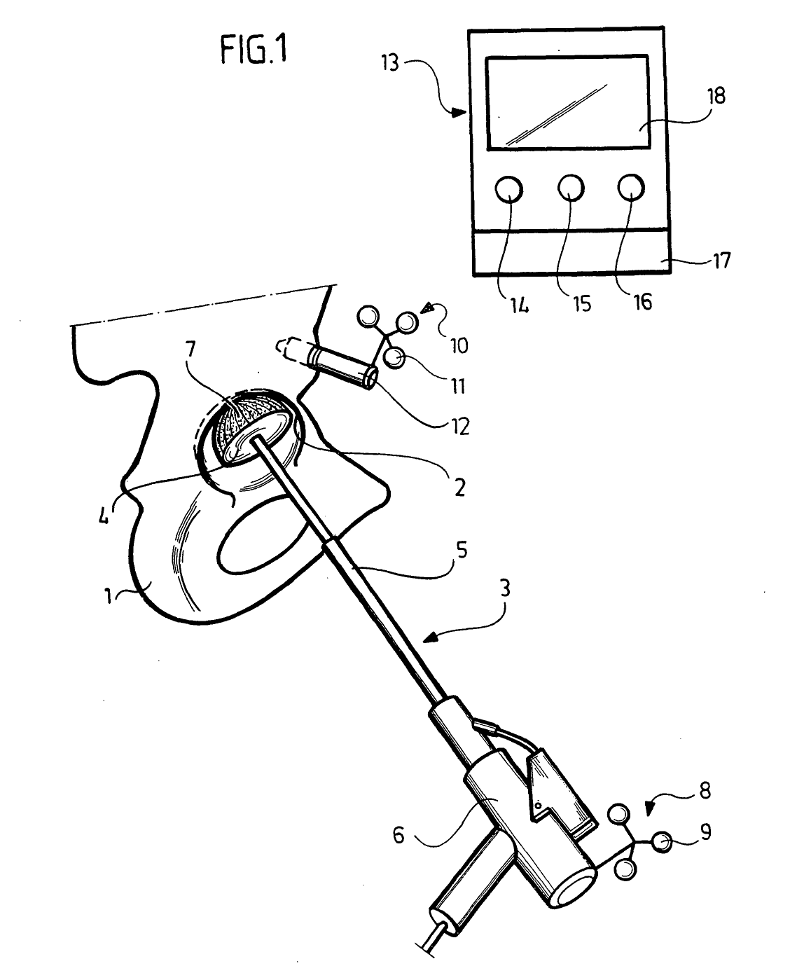

### DESCRIPTION OF THE PREFERRED EMBODIMENTS

The invention is explained below using the example of a hip bone 1, into which is to be worked an approximately hemispherical, cup-shaped recess 2 serving to receive an approximately hemispherical socket of a hip joint implant. This implant is not shown in the drawing. The invention may be applied particularly advantageously in the production of recesses in bone material for surgical purposes, but fundamentally the invention may also be used with other pieces of material, e.g. also in the shaping of implants made of bone material or even other materials, and is therefore not restricted to the machining of pieces of material from the human body or those of animals, even though this field represents a particularly advantageous area of application.

To be able to work the cup-shaped recess 2 into the hip bone, a tool 3 is used which uses an approximately hemispherical profiling cutter 4, which is connected via a rigid shaft 5 to a drive motor, which is accommodated in a handle-type housing 6 of the tool 3. The shaft 5 is set in rotation by the drive and thereby rotates the profiling cutter 3 so that the hemispherical cutting surface 7 of the profiling cutter 4 rotates in itself.

The tool 3 bears a marking element 8 connected rigidly to the housing 6 with three spherical reflection bodies 9 spaced from one another, an identical marking element 10 likewise with three reflection bodies 11 spaced from one another is rigidly connected to the hip bone 1 by means of a bone screw 12 screwed into it.

A navigation system 13 is arranged to be fixed in space and in the shown embodiment has three radiation transmitters and receivers 14, 15, 16, which radiate an ultrared radiation, for example, and receive this again after reflection on the reflection bodies 9 or 11. The transit times of the radiation between the radiation transmitters and receivers 14, 15, 16 and the reflection bodies 9 or 11 of the marking elements 8 and 10 are determined in the arithmetic and memory unit 17 of the navigation system 13, and the precise positions of the marking elements 8 and 10 in space can be determined therefrom, and these positions are unequivocally associated with the positions of the hip bone 1 and the tool 3 in space. In this way, the navigation system 13 can continuously establish the precise positioning both of the hip bone 1 and the tool 3 in space during the entire machining process.

Position data corresponding to these respective positions of the hip bone 1 and the tool 3 are stored in the arithmetic and memory unit 17 so that the entire movement of the hip bone 1 and the tool 3 during the machining process can be reconstructed after conclusion of the machining process.

Moreover, the position data of the hip bone 1 and the tool 3 are compared to one another in the arithmetic and memory unit 17 and from these it is calculated where the cutting surface 7 of the tool 3 is respectively located in relation to the hip bone 1 during the machining. Since the tool 3 is a rigid body and since the cutting surface 7 rotates in itself on rotation of the rigid shaft 5, the envelope formed by the cutting surface 7 is alway the same in relation to the rest of the tool 3 and therefore also in relation to the corresponding marking element 8. Therefore, it is absolutely possible to determine the precise position of this enveloping cutting surface 7 during the entire machining process and to bring it into relation with the position data of the hip bone 1 received via the position of the marking element 10.

Geometric data of the hip bone 1 describing the shape of the hip bone 1, which are received, for example, through a computer tomography image of the hip bone 1, are also made available to the arithmetic and memory unit. In this way, the arithmetic and memory unit 17 can also calculate whether the enveloping cutting surface 7 penetrates into the contour of the hip bone 1, where this occurs exactly and to what extent. In other words, therefore, it is determined which part of the cutting surface 7 has passed through the outer contour of the hip bone 1. This is a measure for indicating the region in which and the extent to which material has been removed from the hip bone 1 by means of the cutting surface 7, the profiling cutter 4 has generated a recess in this region.

The arithmetic and memory unit calculates this penetration of the cutting surface 7 into the contour of the hip bone 1 during the entire machining process and stores these data. At the same time, extreme values of this penetration are established, i.e. the position data corresponding to the deepest penetration of the cutting surface 7 into the contour of the hip bone 1. This can be achieved, for example, by the distances reached at maximum by the cutting surface 7 of the profiling cutter 4 in different directions starting from a firmly assumed point in the hip bone 1, in particular starting from the centre point of a desired semispherical recess 2. If these extreme values are determined in different directions over the entire extension of the desired recess, then position data are obtained which specify the actual boundary wall of the generated recess 2 in the hip bone 1, i.e. the actual contour of the recess 2.

The position data of the actual contour of the recess 2 thus obtained in the arithmetic and memory unit 17 can be visually displayed on a screen 18 of the navigation system 13, so that the operator can read directly on the screen 18 how far the profiling cutter 4 has penetrated into the hip bone 1 and the exact shape that the recess 2 has.

Various display possibilities are given here, e.g. the respectively reached depths of the recess 2 could be displayed directly on the screen in a sectional view, wherein the cutting planes could be varied. It is also possible to superpose the representation of the recess with an image of the hip bone, which had been obtained on the basis of preceding computer tomographic images and stored in the arithmetic and memory unit. In this way, the operator can see directly on an image of the hip bone 1 where and how the recess 2 is being generated by the profiling cutter 4.

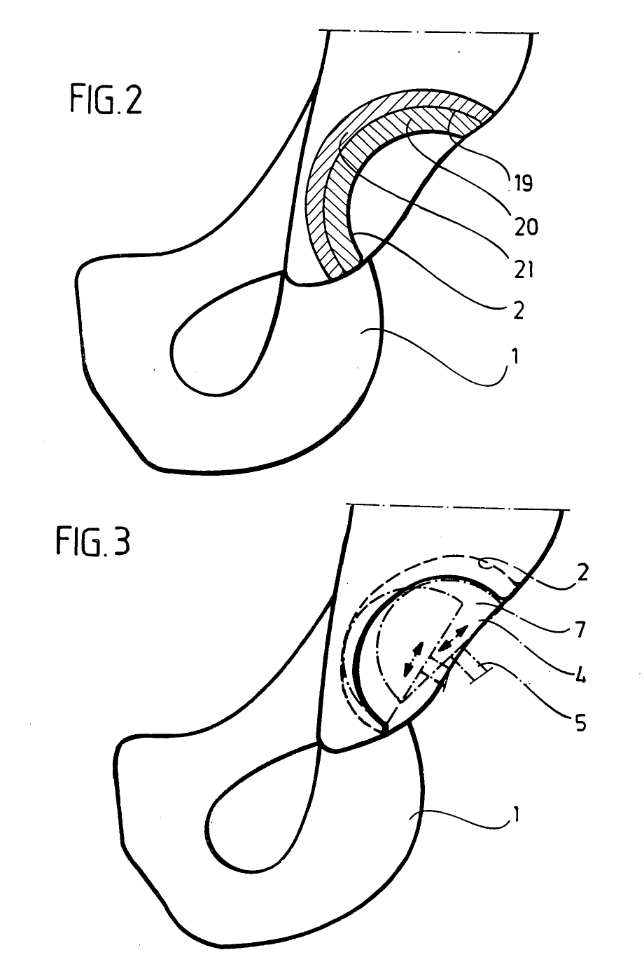

In a preferred embodiment of the invention, the position data of a desired contour of the recess 2 are predetermined and stored in the arithmetic and memory unit. For example, this desired contour can be a hemispherical contour 19, as is shown schematically in the sectional view of FIG. 2. The recess 2 should be machined by the profiling cutter 4 as far as this desired contour 19.

For this, the material of the hip bone 1 is divided into concentric layers, of which only an inside layer 20 and an outside layer 21, which are arranged on opposite sides of the desired contour 19, are indicated in FIG. 2.

By means of the arithmetic and memory unit 17 it is respectively determined over the entire surface of the recess 2 whether the contour actually generated by the profiling cutter 4 lies within the inside layer 20, on the desired contour 19 or in the outside layer 21, and in a view 22 of the spherical recess 2, which can be in two-dimensional or three-dimensional form, the different regions are identified differently in the display, e.g. with different colours. Therefore, regions in which the profiling cutter 4 has not yet reached the inside layer 20 are identified, for example, by a colourless area 23 in view 22, regions in which the cutting surface 7 has advanced into the inside layer 20 are identified by a green area 24 and regions in which the cutting surface 7 has reached the outside layer 21 or has penetrated into this are identified by a red area 25. The operator can therefore see immediately from view 22 where anything has changed in the region of the displayed recess 2 during the machining, this being an indication that precisely in this region machining and deepening of the recess 2 is occurring, and moreover he/she can also see whether the cutting surface 7 is located in the area just machined still outside the inside layer 20, already within the inside layer 20 or even already in the outside layer 21, and accordingly the tool 3 can be guided differently so that the recess 2 can be machined so that the actual recess 2 is optimally matched to the desired contour 19 after the machining process has ended.

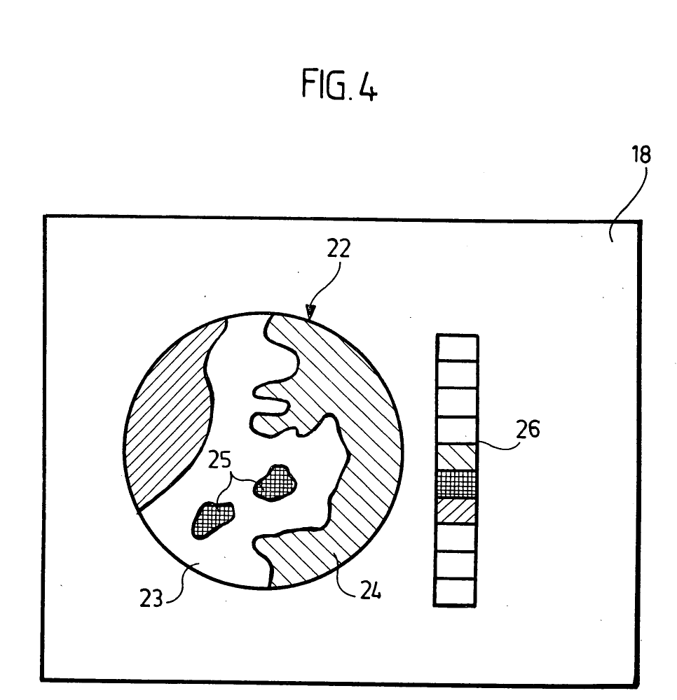

In the representation in FIG. 4, besides the view 22 the respective thickness of the layers 20 and 21 is also given in a scale 26 arranged next to view 22, and naturally the number of corresponding layers can in fact also be substantially higher, so that a very precise removal of the material is possible.

A report of the machining process can be simultaneously created through the position data stored in the arithmetic and memory unit 17 so that after machining of the hip bone 1 has concluded it can be precisely established how the tool 3 had been guided during the machining process and whether the recess 2 fully corresponds to the desired contour 19.

The respectively established position data of the profiling cutter 4 can also be used to prevent an unintentionally deep penetration of the profiling cutter 4 into the hip bone 1. Limit values can be established here for the penetration, and should these be reached in any area of the recess 2, a warning signal or a cutout signal can be generated.

With the described method it is also possible to check, record and when appropriate compensate for unintentional deviating movements of the profiling cutter 4 during machining that can not be avoided in practice, since the user can precisely follow the progress of the machining process on the screen 18 and can guide the tool 3 depending on the progress of the machining process. This is possible despite there being no directly visual access to the machining location, and similarly it is not necessary to create such a view via X-ray observation.

What is claimed is:

1. Method for determining the contour of a recess in a piece of material, which is worked into the material by means of a tool, in particular in a piece of bone, wherein the position of the piece of material in space is established by a navigation system, the position of the tool in space is established by a navigation system, the respective position of the tool in relation to the piece of material is determined from the position data obtained in this manner, during the machining of the piece of material with the tool, the relative positions of the tool in relation to the piece of material are stored and the prepared contour on the piece of material is determined from extreme values of these relative positions with respect to a fixed reference position of the piece of material.

2. Method according to claim 1, wherein the prepared contour is visually displayed.

3. Method according to claim 1, wherein the difference of the contour of the prepared recess determined in this manner from a predetermined contour is determined at different locations of the contour.

4. Method according to claim 3, wherein the differences determined in this manner at different locations of the prepared recess are visually displayed.

5. Method according to claim 4, wherein the same differences at different locations of the prepared recess are displayed in the same manner.

6. Method according to claim 5, wherein the same differences are displayed in the same colour.

7. Method according to claim 2, wherein an image of the piece of material is superposed on the image of the contour of the recess or of the detected difference.

8. Method according to claim 5, wherein an image of the piece of material is superposed on the image of the contour of the recess or of the detected difference.

9. Method according to claim 1, wherein from the determined extreme values of the relative position, a warning signal is generated when these extreme values exceed specific given maximum values.

10. Device for determining the contour of a recess in a piece of material with a tool for working the contour into the piece of material, wherein the piece of material and the tool are respectively firmly connected to a marking element a stationary navigation system is provided, which establishes the position of the two marking elements and thus the position of the piece of material and the tool in space, an arithmetic unit with memory is provided, which from the position data obtained in this manner determines the respective position of the tool in relation to the piece of material, stores the relative positions of the tool in relation to the piece of material during machining of the piece of material by the tool, and determines the prepared contour on the piece of material from extreme values of these relative positions with respect to a fixed reference position of the piece of material.

11. Device according to claim 10, wherein a display device is provided which visually displays the prepared contour.

12. Device according to claim 10, wherein the arithmetic unit with memory determines the difference of the contour of the prepared recess determined in this manner from a predetermined contour at different locations of the contour.

13. Device according to claim 12, wherein the arithmetic unit with memory visually displays the differences determined in this manner at different locations of the prepared recess.

14. Device according to claim 13, wherein the arithmetic unit with memory displays the same differences at different locations of the prepared recess in the same manner.

15. Device according to claim 14, wherein the arithmetic unit with memory displays the same differences in the same colour.

16. Device according to claim 10, wherein the arithmetic unit with memory superposes an image of the piece of material on the image of the contour of the recess or of the detected difference.

17. Device according to claim 12, wherein the arithmetic unit with memory superposes an image of the piece of material on the image of the contour of the recess or of the detected difference.

18. Device according to claim 10, wherein from the determined extreme values of the relative position, the arithmetic unit with memory generates a warning signal when these extreme values exceed specific given maximum values.
